(19) United States
(12) Patent Application Publication
(10) Pub. No.: US 2026/0257020 A1
(43) Pub. Date: Sep. 3, 2026

(54) Improved feedback for rechargeable medication delivery device

(71) Applicant: SHL Medical AG, Zug (CH)

(72) Inventors: Alejandra Linares Martinez, Boonton, NJ (US); James Thomas Hawthorne, Boonton, NJ (US); Martin Michael Coyne, III, Boonton, NJ (US)

(21) Appl. No.: 19/155,819

(22) PCT Filed: Feb. 12, 2024
(86) PCT No.: PCT/EP2024/053467

§ 371 Date: Aug. 12, 2025

Related U.S. Application Data
(60) Provisional application No. 63/447,662, filed on Feb. 23, 2023.

(30) Foreign Application Priority Data
Mar. 21, 2023 (EP) 23163003.9

(51) Int. Cl. A61M 5/315

(52) U.S. Cl. CPC A61M 5/31576; A61M 2005/31588; A61M 2205/502; A61M 2205/8206; A61M 2205/8256

(57) ABSTRACT

The present disclosure provides a reusable drive unit for use with a medication delivery device. The reusable drive unit includes a drive mechanism, a battery configured to supply power to the drive mechanism, at least one indicator icon, at least one memory storage element, at least one processor, and data storage including program instructions stored thereon that when executed by the at least one processor, cause the reusable drive unit to perform functions. The functions include (i) perform one or more injections of a dose interval of a medicament to a patient via one or more disposable drug units removably coupled to the reusable drive unit, (ii) store, in the at least one memory storage element, a power requirement profile for the one or more injections, and (iii) display, via the at least one indicator icon, a number of injections possible based on a current charge level of the battery and the stored power requirement profile.

### TECHNICAL FIELD

The present disclosure generally relates to medication delivery devices, and more particularly to an improved indication of the charge level of reusable battery driven medication delivery devices.

### BACKGROUND

Reusable medication delivery devices offer improved clinical flexibility and environmental sustainability. Such devices typically have a reusable drive unit with a drive mechanism, a battery, and supporting circuits, intended for use with a disposable drug unit containing one or more doses of medication. Patients store the reusable component in between medication administration intervals. The reusable component also typically contains indicators to provide users feedback on the device readiness, injection process, and injection feedback.

However, the advent of reusable medication delivery devices presents several challenges. Reusable drive units require periodic recharging. Confusion may arise if the same visual indicator is used for dose delivery progress and charging status. If different indicators are used, usability may improve, but device complexity (and cost) increases, as more indicators are needed in/on the device. Thus, improved solutions are needed to allow a user to unambiguously discern injection and charge progression, particularly based on the use context of the device—charging, injection, or some other state.

Additionally, multiple injections are possible with reusable devices. For instance, with a 5 mL drug container and a 10 mL dose per interval, two drug containers are required, each administered sequentially. Similarly, 15 mL could be delivered with three drug containers sequentially, each containing a familiar glass cartridge or syringe. This approach may be an alternative to more complex on-body injectors (OBIs) that deliver larger doses with a single device and primary container which may be less familiar and require new stability studies. When a reusable device is used in the context of multiple sequential injections, it is highly desirable (if not mandatory) that the device has sufficient charge to administer all the injections required for a single dosing interval. However, conventional charging indicators may not communicate this well to a user, who may not be able to discern whether a charge level (e.g., 50%) permits all the injections in an interval to be administered.

Further, the temperature and viscosity of the medicament to be administered affects this determination. Generally speaking, more viscous medications require higher motor load, and thus higher power requirements and higher charge states to perform an injection. Higher viscosity may be a function of the temperature or concentration of a medicament (biologics, for instance, have a nonlinear viscosity-temperature-concentration relationship. For example, a 25% charge may be enough to inject one dose at room temperature but not enough charge at a refrigerated temperature.

Thus, improved solutions are needed to allow a user to easily interpret whether a multiple drug container administration may be performed by the device.

### SUMMARY

Principles and embodiments of the present invention relate to improvements in charging and monitoring the charge level of reusable battery driven medication delivery devices. An improved method of user feedback is provided, wherein the charge level is expressed either as integer number of injections available based on the charge level, or a portion thereof. Embodiments of the apparatus may be used on reusable autoinjectors, subcutaneous infusion pumps, intravenous pumps, intrathecal pumps, insulin pumps, large-volume infusers, or devices for delivery of medicaments by other routes of administration.

In particular, in a first aspect, the present disclosure provides a reusable drive unit for use with a medication delivery device. The reusable drive unit includes a drive mechanism, a battery configured to supply power to the drive mechanism, at least one indicator icon, at least one memory storage element, at least one processor, and data storage including program instructions stored thereon that when executed by the at least one processor, cause the reusable drive unit to perform functions. The functions include (i) perform one or more injections of a dose interval of a medicament to a patient via one or more disposable drug units removably coupled to the reusable drive unit, (ii) store, in the at least one memory storage element, a power requirement profile for the one or more injections, and (iii) display, via the at least one indicator icon, a number of injections possible based on a current charge level of the battery and the stored power requirement profile.

In one embodiment of the first aspect, the one or more injections of the dose interval of the medicament comprises at least two injections from at least two disposable drug units removably coupled to the reusable drive unit in sequence.

In another embodiment of the first aspect, the at least one indicator icon comprises at least three indicator icons.

In another embodiment of the first aspect, the at least one indicator icon comprises a graphical indication of the number of injections possible based on the current charge level of the battery and the stored power requirement profile.

In another embodiment of the first aspect, the at least one indicator icon comprises a numerical indication of the number of injections possible based on the current charge level of the battery and the stored power requirement profile.

In another embodiment of the first aspect, the reusable drive unit further includes a charging case configured to receive the reusable drive unit, wherein charging case includes at least one indicia configured to interact with the at least one indicator icon to display the number of injections possible based on the current charge level of the battery and the stored power requirement profile.

In another embodiment of the first aspect, light from the at least one indicator icon is in optical communication with the at least one indicia of the charging case to thereby display the number of injections possible based on the current charge level of the battery and the stored power requirement profile.

In another embodiment of the first aspect, the number of injections possible based on the current charge level of the battery and the stored power requirement profile is displayed in both right reading and left reading orientations.

In another embodiment of the first aspect, the power requirement profile includes a temperature measurement of the medicament and/or a temperature measurement of the battery.

In another embodiment of the first aspect, the program instructions further cause the reusable drive unit to adjust the display of the number of injections possible based on at least one of a current temperature measurement of the medicament or a current temperature measurement of the battery.

In another embodiment of the first aspect, the program instructions further cause the reusable drive unit to identify the medicament in the one or more disposable drug units removably coupled to the reusable drive unit, and adjust the display of the number of injections possible based on a current identification of the medicament in the one or more disposable drug units removably coupled to the reusable drive unit.

In a second aspect, the current disclosure provides a method for providing feedback for a rechargeable medication delivery device, the method comprising: (i) removably coupling one or more disposable drug units to a reusable drive unit of the medication delivery device, (ii) performing one or more injections of a dose interval of a medicament to a patient via the one or more disposable drug units removably coupled to the reusable drive unit, (iii) storing, in at least one memory storage element of the reusable drive unit, a power requirement profile for the one or more injections, and (iv) displaying, via at least one indicator icon of the reusable drive unit, a number of injections possible based on a current charge level of a battery of the reusable drive unit and the stored power requirement profile.

In one embodiment of the second aspect, the power requirement profile includes a temperature measurement of the medicament and/or a temperature measurement of the battery.

In another embodiment of the second aspect, the method further includes adjusting the display of the number of injections possible based on at least one of a current temperature measurement of the medicament or a current temperature measurement of the battery.

In another embodiment of the second aspect, the method further includes identifying the medicament in the one or more disposable drug units removably coupled to the reusable drive unit.

In another embodiment of the second aspect, the method further includes adjusting the display of the number of injections possible based on a current identification of the medicament in the one or more disposable drug units removably coupled to the reusable drive unit.

In another embodiment of the second aspect, the one or more injections of the dose interval of the medicament comprises at least two injections from at least two disposable drug units removably coupled to the reusable drive unit in sequence.

In another embodiment of the second aspect, the at least one indicator icon comprises at least three indicator icons.

In another embodiment of the second aspect, the at least one indicator icon comprises a graphical indication of the number of injections possible based on the current charge level of the battery and the stored power requirement profile.

In another embodiment of the second aspect, the at least one indicator icon comprises a numerical indication of the number of injections possible based on the current charge level of the battery and the stored power requirement profile.

In another embodiment of the second aspect, at last a portion of the at least one indicator icon contains a graphic representation of the medication delivery device.

These as well as other aspects, advantages, and alternatives, will become apparent to those of ordinary skill in the art by reading the following detailed description, with reference where appropriate to the accompanying drawings.

### BRIEF DESCRIPTION OF THE FIGURES

FIG. 1 illustrates a simplified block diagram of a medication delivery device, according to an example embodiment.

FIG. 2A illustrates indicator icons positioned on the reusable drive unit, according to an example embodiment.

FIG. 2B illustrates indicator icons positioned on the reusable drive unit, according to an example embodiment.

FIG. 3A illustrates indicator icons positioned on a charging case of the reusable drive unit, according to an example embodiment.

FIG. 3B illustrates indicator icons positioned on a charging case of the reusable drive unit, according to an example embodiment.

FIG. 3C illustrates indicator icons positioned on a charging case of the reusable drive unit, according to an example embodiment.

FIG. 4 illustrates indicator icons positioned on the reusable drive unit, according to an example embodiment.

FIG. 5 is a flowchart illustrating an iterative process for adjusting a display of one or more indicator icons of the reusable drive unit, according to an example embodiment.

FIG. 6 is a block diagram of a method for providing feedback for a rechargeable medication delivery device, according to an example embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The example embodiments described herein are not meant to be limiting. It will be readily understood that the aspects of the present disclosure, as generally described herein, and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an example embodiment may include elements that are not illustrated in the Figures.

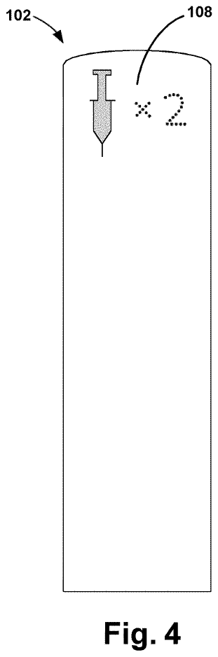

In FIG. 4, referred to above, the blocks may represent operations and/or portions thereof and lines connecting the various blocks do not imply any particular order or dependency of the operations or portions thereof. It will be understood that not all dependencies among the various disclosed operations are necessarily represented. FIG. 4 and the accompanying disclosure describing the operations of the method(s) set forth herein should not be interpreted as necessarily determining a sequence in which the operations are to be performed. Rather, although one illustrative order is indicated, it is to be understood that the sequence of the operations may be modified when appropriate. Accordingly, certain operations may be performed in a different order or simultaneously. Additionally, those skilled in the art will appreciate that not all operations described need be performed.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, apparatus, element and method “configured to” perform a specified function is indeed capable of performing the specified function without any alteration, rather than merely having potential to perform the specified function after further modification. In other words, the apparatus, element, and method “configured to” perform a specified function is specifically selected, created, implemented, utilized, programmed, and/or designed for the purpose of performing the specified function. As used herein, “configured to” refers to existing characteristics of an apparatus, element, and method which enable the apparatus, element, and method to perform the specified function without further modification. For purposes of this disclosure, an apparatus, element, and method described as being “configured to” perform a particular function can additionally or alternatively be described as being “adapted to” and/or as being “operative to” perform that function.

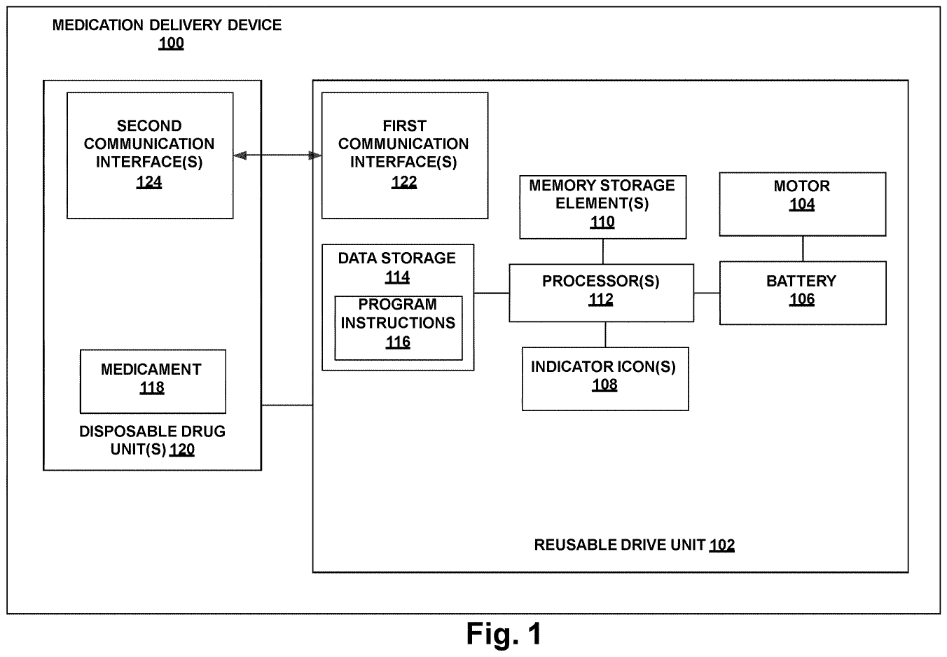

With reference to the figures, FIG. 1 illustrates a simplified block diagram of a medication delivery device 100, according to an example embodiment. As shown in FIG. 1, the medication delivery device 100 includes a reusable drive unit 102 for use with a medication delivery device 100. The reusable drive unit 102 includes a drive mechanism 104 and a battery 106 configured to supply power to the drive mechanism 104. The drive mechanism 104 may comprise a motor, a fluid pump, or a pressure-driven system, as non-limiting examples. The reusable drive unit 102 further includes at least one indicator icon 108 as discussed in additional detail below.

The reusable drive unit 102 further includes at least one memory storage element 110. The at least one memory storage element 110 can include any type of memory now known or later developed including but not limited to volatile memory (such as RAM), non-volatile memory (such as ROM, flash memory, etc.) or any combination thereof. The reusable drive unit 102 also includes at least one processor 112 and data storage 114 including program instructions 116 stored thereon that when executed by the at least one processor 112, cause the reusable drive unit 102 to perform functions. Although various components of the medication delivery device 100 are shown as distributed components, it should be understood that any of such components may be physically integrated and/or distributed according to the desired configuration of the system. Depending on the desired configuration, the at least one processor 112 can be any type of processor including, but not limited to, a microprocessor, a microcontroller, a digital signal processor, or any combination thereof.

In particular, the functions include (i) perform one or more injections of a dose interval of a medicament 118 to a patient via one or more disposable drug units 120 removably coupled to the reusable drive unit 102, (ii) store, in the at least one memory storage element 110, a power requirement profile for the one or more injections, and (iii) display, via the at least one indicator icon 108, a number of injections possible based on a current charge level of the battery 106 and the stored power requirement profile.

In one example, the power requirement profile includes a temperature measurement of the medicament 118 and/or a temperature measurement of the battery 106. In such an example, the program instructions 116 further cause the reusable drive unit 102 to adjust the display of the number of injections possible based on at least one of a current temperature measurement of the medicament 118 or a current temperature measurement of the battery 106.

In another example, the program instructions 116 further cause the reusable drive unit 102 to (i) identify the medicament 118 in the one or more disposable drug units 120 removably coupled to the reusable drive unit 102, and adjust the display of the number of injections possible based on a current identification of the medicament 118 in the one or more disposable drug units 120 removably coupled to the reusable drive unit 102.

In one example, the reusable drive unit 102 further include at least one first communication interface 122 configured to receive information from at least one second communication interface 124 of the disposable drug unit 120 when the disposable drug unit 120 is removably coupled to the reusable drive unit 102. Such information may include an identification and/or characteristics of the medicament 118 in the disposable drug unit 120. The at least one first communication interface 122 and the at least one second communication interface 124 may contain hardware to enable a communication link therebetween, such as processors, transmitters, receivers, antennas, etc., as discussed above. In one example, the communication link between the at least one first communication interface 122 and the at least one second communication interface 124 is a wired connection. In another example, the communication link between the at least one first communication interface 122 and the at least one second communication interface 124 is a wireless connection such as radio frequency identification (RFID), near-field communication (NFC), Bluetooth, Bluetooth low energy (BLE), Ultra wide band (UWB), wireless fidelity (Wi-Fi), cellular communication, and infrared (IR), as non-limiting examples.

In one example, the one or more injections of the dose interval of the medicament 118 comprises at least two injections from at least two disposable drug units 120 removably coupled to the reusable drive unit 102 in sequence.

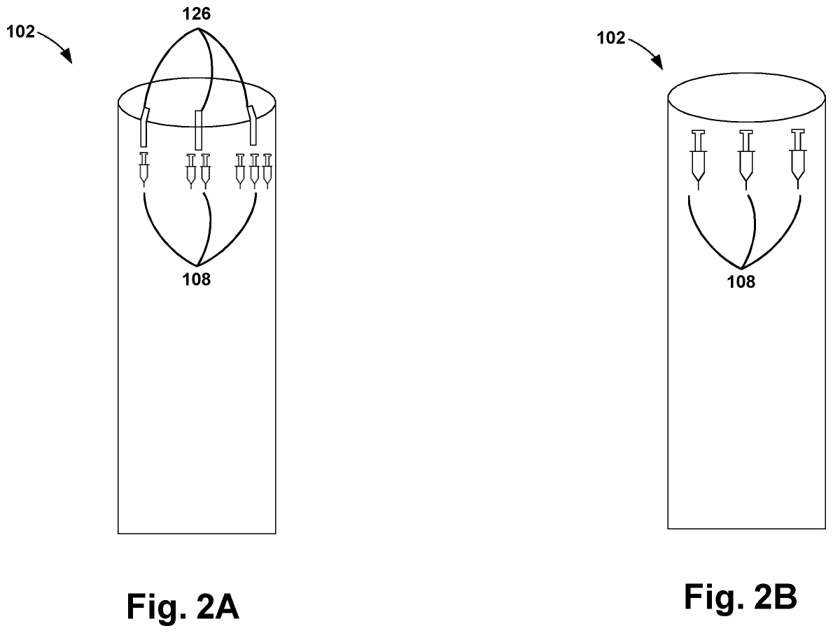

In one specific example, as shown in FIGS. 2A-2B, the at least one indicator icon 108 comprises at least three indicator icons. As further shown in FIGS. 2A-2B, the at least one indicator icon 108 comprises a graphical indication of the number of injections possible based on the current charge level of the battery and the stored power requirement profile. FIG. 2A shows one embodiment including LED segments 126. The LED segments 126 may be used to provide injection feedback, and the additional indicator icons 108 are used to provide charging status corresponding to the number of injections possible based on the current charge state of the reusable drive unit 102. The indicator icons 108 shown in FIG. 2A may be printed, debossed, or also light up.

FIG. 2B shows an alternate embodiment indicator icons 108 that can be illuminated as injection process feedback LEDs during injection, and during charging, the indicator icons 108 may be illuminated in groups corresponding to the number of injections possible based on the current charge state of the reusable drive unit 102. More specifically, the charge status displays of FIGS. 2A-2B, correspond not to a charge percentage or level (e.g., 0%, 10%, 50%, 100% etc.) but groups of injections possible based on the charge state of the device (e.g., 1, 2, or 3 injections). If the charge level is insufficient for even a single injection, no icons may be illuminated. If the charge level is comparatively low but sufficient to complete one injection, a single injection icon may be shown. This may be extended to 2, 3, or other number of discrete injections. In preferred embodiments, at least a portion of the at least one indicator icon 108 contains a graphic representation (e.g., an outline as a non-limiting example) of the medication delivery device 100 being used for the injection.

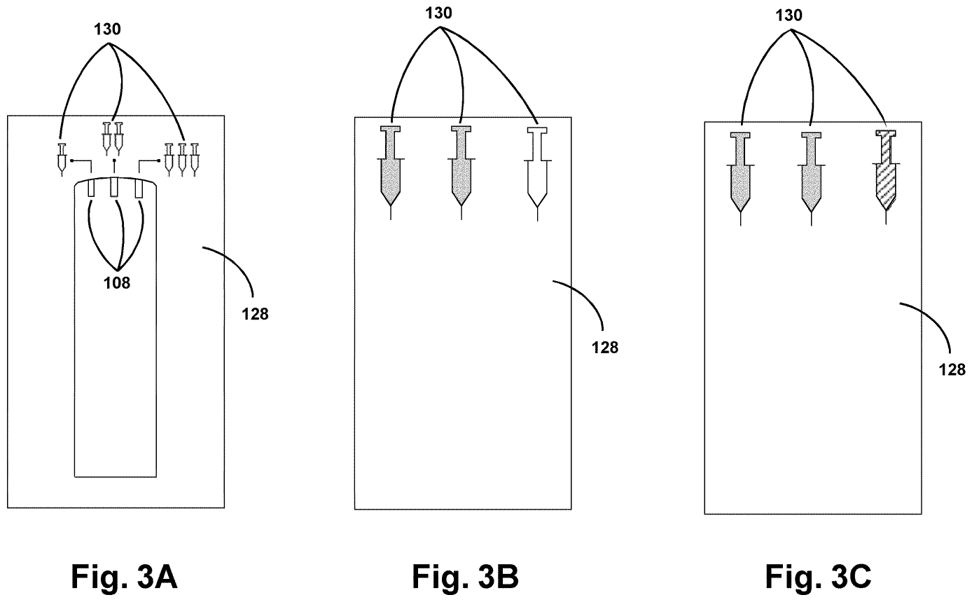

In another example, as shown in FIGS. 3A-3C, a charging case 128 is configured to receive the reusable drive unit 102 while the reusable drive unit 102 is charging the battery 106. In such an example, the charging case 128 may include at least one indicia 130 configured to interact with the at least one indicator icon 108 of the reusable drive unit 102 to display the number of injections possible based on the current charge level of the battery 106. and the stored power requirement profile. Such an arrangement, may be optionally provided with at least one indicia 130 to aid in a user's interpretation of the charging state, as shown in FIG. 3A.

In another example, as shown in FIGS. 3B-3C, light from the at least one indicator icon 108 of the reusable drive unit 102 is in optical communication with the at least one indicia 130 of the charging case 128 to thereby display the number of injections possible based on the current charge level of the battery 106 and the stored power requirement profile. In one example, light from the at least one indicator icon 108 of the reusable drive unit 102 passes through a light pipe to the at least one indicia 130 of the charging case 128. In one example, there may be one light source per indicator icon 108. Other arrangements are possible as well.

In one example, as shown in FIG. 3B, the at least one indicia 130 of the charging case 128 may be dimmed entirely when charging is in progress (illustrating charging in process for a 3^{rd} injection, with two injections “ready to go”). In another example, as shown in FIG. 3C, the at least one indicia 130 of the charging case 128 may be provided with a different color when charging is in progress (illustrating charging in process for a 3^{rd} injection, with two injections “ready to go”).

While previous illustrative embodiments show use of iconography to indicate the number of injections the reusable drive unit 102 is capable of delivering, FIG. 4 shows an alternative embodiment in which the at least one indicator icon 108 comprises a numerical indication of the number of injections possible based on the current charge level of the battery 106 and the stored power requirement profile. In this instance, an icon may be provided (again, reflecting the shape of the device or delivery route) and a lit segment display can be used to indicate the number of injections that may be delivered based on the current charge state. FIG. 4A shows, by way of example, a charge state corresponding to two injections. This indicator design has the additional advantage that when unlit, the numeric indicator is invisible to the user (only the syringe icon of FIG. 4A would be visible in this context). In one example, the number of injections possible based on the current charge level of the battery 106 and the stored power requirement profile is displayed in both right reading and left reading orientations.

One primary advantage of reusable devices is ability to deliver medications of widely varying viscosities, owing to electronic control and drive mechanisms (e.g., pumps, motors, etc.). Generally speaking, more viscous medications require higher motor load, and thus higher power requirements and higher charge states. Higher viscosity may be a function of the temperature or concentration of a medicament (biologics, for instance, have a nonlinear viscosity-temperature-concentration relationship). Another advantage of reusable devices is that the reusable drive may be programmed by a drug cassette or other drug container, as by use of the first communication interface 122 on the reusable drive unit 102 and the second communication interface 124 on the disposable drug unit(s) 120 (see e.g., WO2018/010931, WO2020/064339, and EP3639871). Such programming may include, by way of example, the medication, concentration, dosing regimen, number of drug containers, or other relevant factors for a specific medication.

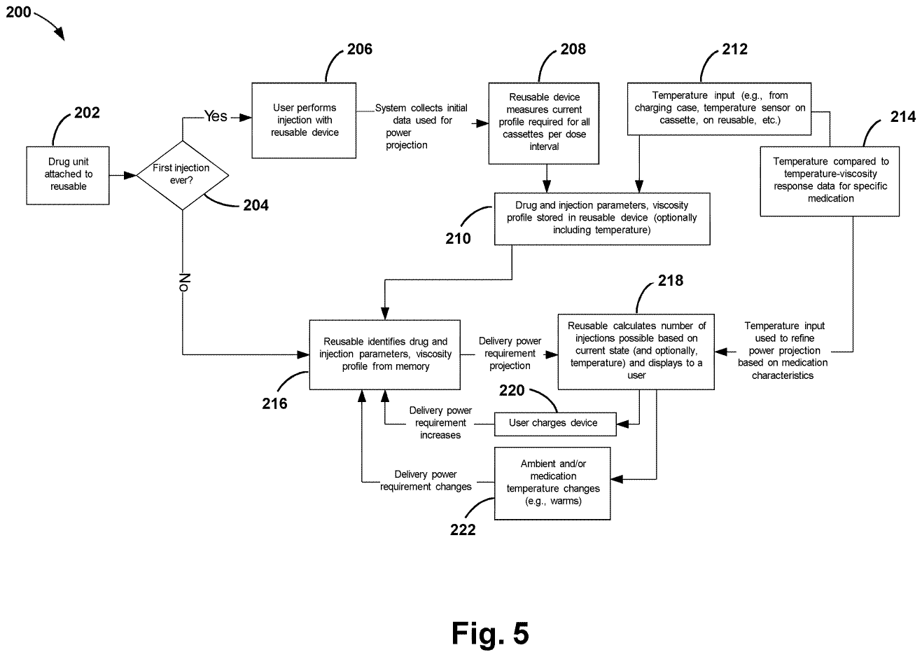

This may be advantageously combined with the present disclosure to adjust the charge feedback based on the expected load placed on the reusable drive unit 102 and adjust the charging feedback provided to a user. FIG. 5 illustrates a method by which the indicators may be adapted to the power requirements of a specific medication based on viscosity, typical injection temperatures, or other factors.

FIG. 5 illustrates an example method 200. With reference to FIG. 5, at block 202 a disposable drug unit 120 is attached to the reusable drive unit 102. At block 204, the reusable drive unit 102 determines whether or not this is a first injection for the reusable drive unit 102 (e.g., it is the first ever injection for the reusable drive unit 102 there is no stored profile in the medication delivery device 100 for that particular patient). If it is not a first injection, the method 200 continues at block 216. If the reusable drive unit 102 determines that it is a first injection, the method 200 continues at block 206 with performing one or more injections of a dose interval with the reusable drive unit 102. At block 208, the reusable drive unit 102 determines a power requirement profile for the one or more injections of the dose interval. As shown at block 210, the power requirement profile may include drug and injection parameters, viscosity profile, and temperature data that are stored in the at least one memory storage element 110 of the reusable drive unit 102. As shown in block 212, the temperature input may be from the charging case 128, a temperature sensor on the reusable drive unit 102, or a temperature sensor on the disposable drug unit 120, or combinations thereof, all as non-limiting examples. As shown in block 214, the measured temperature may be compared to a temperature-viscosity profile for a known medicament. At block 216, the reusable drive unit 102 identifies the medicament 118 in the disposable drug unit 120 (e.g., via a NFC) and recalls the power requirement profile from the at least one memory storage element 110 of the reusable drive unit 102. At block 218, the reusable drive unit 102 uses the power requirement profile and the current charge state of the battery 106 to calculate a number of injections possible based on the current charge state. This may be combined with a mapping of the time-concentration-temperature relationship for a specific medicament (which is verified in the first and following instances by use of the identifying NFC chip or other second communication interface 124 on the disposable drug unit(s) 120). As shown in blocks 220 and 222, the reusable drive unit 102 (or the charging case 128 for the reusable drive unit 102) may also respond to changes in charging state or ambient temperature to further present a refined estimate of charging state to a user of the reusable drive unit 102.

By use of this method, it is possible to allow a user to see if injection is possible, as the reusable drive unit 102 “learns” what the power requirements are for a given user and typical injection routine and/or medication regimen and can provide adaptive feedback. For instance, if a medicament 118 is removed from the fridge cold (and thus is viscous) the reusable drive unit 102 may indicate an injection is not possible until the medicament 118 warms up (or the device is charged more).

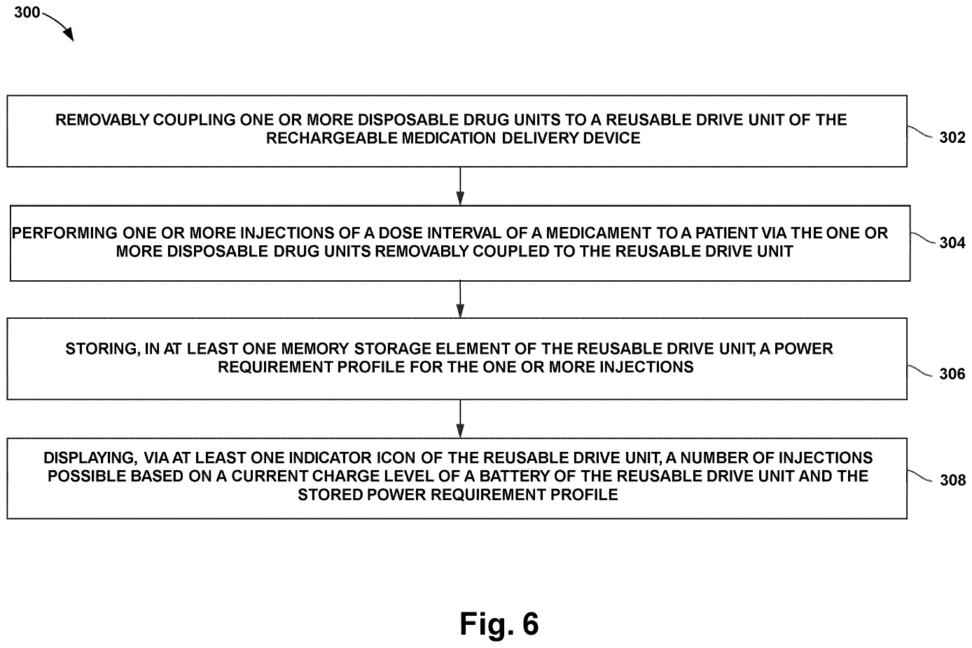

FIG. 6 is a block diagram of an example method for providing feedback for a rechargeable medication delivery device. Method 300 shown in FIG. 6 presents an embodiment of a method that could be used by the medication delivery device 100 as described in FIGS. 1-5, as examples. Method 300 may include one or more operations, functions, or actions as illustrated by one or more of blocks 202-212. Although the blocks are illustrated in a sequential order, these blocks may also be performed in parallel, and/or in a different order than those described herein. Also, the various blocks may be combined into fewer blocks, divided into additional blocks, and/or removed based upon the desired implementation.

In addition, for the method 300 and other processes and methods disclosed herein, the block diagram shows functionality and operation of one possible implementation of present embodiments. In this regard, each block may represent a module, a segment, or a portion of program code, which includes one or more instructions executable by a processor or computing device for implementing specific logical functions or steps in the process. The program code may be stored on any type of computer readable medium, for example, such as a storage device including a disk or hard drive. The computer readable medium may include non-transitory computer readable medium, for example, such as computer-readable media that stores data for short periods of time like register memory, processor cache and Random Access Memory (RAM). The computer readable medium may also include non-transitory media, such as secondary or persistent long term storage, like read only memory (ROM), optical or magnetic disks, compact-disc read only memory (CD-ROM), for example. The computer readable media may also be any other volatile or non-volatile storage systems. The computer readable medium may be considered a computer readable storage medium, for example, or a tangible storage device.

Initially, at block 302, the method 300 includes removably coupling one or more disposable drug units 120 to a reusable drive unit 102 of the rechargeable medication delivery device 100. At block 304, the method 300 includes performing one or more injections of a dose interval of a medicament 118 to a patient via the one or more disposable drug units 120 removably coupled to the reusable drive unit 102. At block 306, the method 300 includes storing, in at least one memory storage element 110 of the reusable drive unit 102, a power requirement profile for the one or more injections. At block 308, the method 300 includes displaying, via at least one indicator icon 108 of the reusable drive unit 102, a number of injections possible based on a current charge level of a battery 106 of the reusable drive unit 102 and the stored power requirement profile.

In one example, the power requirement profile includes a temperature measurement of the medicament 118 and/or a temperature measurement of the battery 106.

In one example, the method 300 further includes adjusting the display of the number of injections possible based on at least one of a current temperature measurement of the medicament 118 or a current temperature measurement of the battery 106.

In another example, the method 300 further includes providing for display of a view of warmup rate (e.g., a time until the medicament 118 is warm enough to inject) based on at least one of a current temperature measurement of the medicament 118 or a current temperature measurement of the battery 106.

In one example, the method 300 further includes identifying the medicament 118 in the one or more disposable drug units 120 removably coupled to the reusable drive unit 102.

In one example, the method 300 further includes adjusting the display of the number of injections possible based on a current identification of the medicament 118 in the one or more disposable drug units 120 removably coupled to the reusable drive unit 102.

In one example, the one or more injections of the dose interval of the medicament 118 comprises at least two injections from at least two disposable drug units 120 removably coupled to the reusable drive unit 102 in sequence. In another example, the at least one indicator icon 108 comprises at least three indicator icons. In another example, the at least one indicator icon 108 comprises a graphical indication of the number of injections possible based on the current charge level of the battery 106 and the stored power requirement profile. In another example, the at least one indicator icon 108 comprises a numerical indication of the number of injections possible based on the current charge level of the battery 106 and the stored power requirement profile.

It will be appreciated that other arrangements are possible as well, including some arrangements that involve more or fewer steps than those described above, or steps in a different order than those described above.

While various aspects and embodiments have been disclosed herein, other aspects and embodiments will be apparent to those skilled in the art. All embodiments within and between different aspects of the devices and methods can be combined unless the context clearly dictates otherwise. The various aspects and embodiments disclosed herein are for purposes of illustration and are not intended to be limiting, with the true scope and spirit being indicated by the claims. Some aspects of the invention are summarized in the following clauses.
1. A reusable drive unit for use with a medication delivery device, the reusable drive unit comprising:
a drive mechanism;
a battery configured to supply power to the drive mechanism;
at least one indicator icon;
at least one memory storage element;
at least one processor; and
data storage including program instructions stored thereon that when executed by the at least one processor, cause the reusable drive unit to:
   perform one or more injections of a dose interval of a medicament to a patient via one or more disposable drug units removably coupled to the reusable drive unit;
   store, in the at least one memory storage element, a power requirement profile for the one or more injections; and
   display, via the at least one indicator icon, a number of injections possible based on a current charge level of the battery and the stored power requirement profile.
2. The reusable drive unit of clause 1, wherein the one or more injections of the dose interval of the medicament comprises at least two injections from at least two disposable drug units removably coupled to the reusable drive unit in sequence.
3. The reusable drive unit of any one of clauses 1-2, wherein the at least one indicator icon comprises at least three indicator icons.
4. The reusable drive unit of any one of clauses 1-3, wherein the at least one indicator icon comprises a graphical indication of the number of injections possible based on the current charge level of the battery and the stored power requirement profile.
5. The reusable drive unit of any one of clauses 1-3, wherein the at least one indicator icon comprises a numerical indication of the number of injections possible based on the current charge level of the battery and the stored power requirement profile.
6. The reusable drive unit of any one of clauses 1-5, further comprising:
a charging case configured to receive the reusable drive unit, wherein charging case includes at least one indicia configured to interact with the at least one indicator icon to display the number of injections possible based on the current charge level of the battery and the stored power requirement profile.
7. The reusable drive unit of clause 6, wherein light from the at least one indicator icon is in optical communication with the at least one indicia of the charging case to thereby display the number of injections possible based on the current charge level of the battery and the stored power requirement profile.
8. The reusable drive unit of any one of clauses 1-7, wherein the number of injections possible based on the current charge level of the battery and the stored power requirement profile is displayed in both right reading and left reading orientations.
9. The reusable drive unit of any one of clauses 1-8, wherein the power requirement profile includes a temperature measurement of the medicament and/or a temperature measurement of the battery.
10. The reusable drive unit of clause 9, wherein the program instructions further cause the reusable drive unit to:
adjust the display of the number of injections possible based on at least one of a current temperature measurement of the medicament or a current temperature measurement of the battery.
11. The reusable drive unit of any one of clauses 1-9, wherein the program instructions further cause the reusable drive unit to:
identify the medicament in the one or more disposable drug units removably coupled to the reusable drive unit; and
adjust the display of the number of injections possible based on a current identification of the medicament in the one or more disposable drug units removably coupled to the reusable drive unit.
12. A method for providing feedback for a rechargeable medication delivery device, the method comprising:
removably coupling one or more disposable drug units to a reusable drive unit of the rechargeable medication delivery device;
performing one or more injections of a dose interval of a medicament to a patient via the one or more disposable drug units removably coupled to the reusable drive unit;
storing, in at least one memory storage element of the reusable drive unit, a power requirement profile for the one or more injections; and
displaying, via at least one indicator icon of the reusable drive unit, a number of injections possible based on a current charge level of a battery of the reusable drive unit and the stored power requirement profile.
13. The method of clause 12, wherein the power requirement profile includes a temperature measurement of the medicament and/or a temperature measurement of the battery.
14. The method of clause 13, further comprising:
adjusting the display of the number of injections possible based on at least one of a current temperature measurement of the medicament or a current temperature measurement of the battery.
15. The method of any one of clauses 12-14, further comprising:
identifying the medicament in the one or more disposable drug units removably coupled to the reusable drive unit.
16. The method of clause 15, further comprising:
adjusting the display of the number of injections possible based on a current identification of the medicament in the one or more disposable drug units removably coupled to the reusable drive unit.
17. The method of any one of clauses 12-16, wherein the one or more injections of the dose interval of the medicament comprises at least two injections from at least two disposable drug units removably coupled to the reusable drive unit in sequence.
18. The method of any one of clauses 12-17, wherein the at least one indicator icon comprises at least three indicator icons.
19. The method of any one of clauses 12-18, wherein the at least one indicator icon comprises a graphical indication of the number of injections possible based on the current charge level of the battery and the stored power requirement profile.
20. The method of any one of clauses 12-19, wherein the at least one indicator icon comprises a numerical indication of the number of injections possible based on the current charge level of the battery and the stored power requirement profile.
21. The method of any one of clauses 12-20, wherein at least a portion of the at least one indicator icon contains a graphic representation of the medication delivery device.

1-15. (canceled)

16. A reusable drive unit for use with a medication delivery device, the reusable drive unit comprising:
a drive mechanism;
a battery configured to supply power to the drive mechanism;
at least one indicator icon;
at least one memory storage element;
at least one processor; and
data storage including program instructions stored thereon that when executed by the at least one processor, cause the reusable drive unit to:
perform one or more injections of a dose interval of a medicament to a patient via one or more disposable drug units removably coupled to the reusable drive unit;
store, in the at least one memory storage element, a power requirement profile for the one or more injections; and
display, via the at least one indicator icon, a number of injections possible based on a current charge level of the battery and the stored power requirement profile.

17. The reusable drive unit of claim 16, wherein the one or more injections of the dose interval of the medicament comprises at least two injections from at least two disposable drug units removably coupled to the reusable drive unit in sequence.

18. The reusable drive unit of claim 16, wherein the at least one indicator icon comprises at least three indicator icons.

19. The reusable drive unit of claim 16, wherein the at least one indicator icon comprises a graphical indication of the number of injections possible based on the current charge level of the battery and the stored power requirement profile.

20. The reusable drive unit of claim 16, wherein the at least one indicator icon comprises a numerical indication of the number of injections possible based on the current charge level of the battery and the stored power requirement profile.

21. The reusable drive unit of claim 16, further comprising:
a charging case configured to receive the reusable drive unit, wherein charging case includes at least one indicia configured to interact with the at least one indicator icon to display the number of injections possible based on the current charge level of the battery and the stored power requirement profile.

22. The reusable drive unit of claim 21, wherein light from the at least one indicator icon is in optical communication with the at least one indicia of the charging case to thereby display the number of injections possible based on the current charge level of the battery and the stored power requirement profile.

23. The reusable drive unit of claim 16, wherein the number of injections possible based on the current charge level of the battery and the stored power requirement profile is displayed in both right reading and left reading orientations.

24. The reusable drive unit of claim 16, wherein the power requirement profile is based on a temperature measurement of the medicament and/or a temperature measurement of the battery.

25. The reusable drive unit of claim 24, wherein the program instructions further cause the reusable drive unit to:
adjust the display of the number of injections possible based on at least one of a current temperature measurement of the medicament or a current temperature measurement of the battery.

26. The reusable drive unit of claim 16, wherein the program instructions further cause the reusable drive unit to:
identify the medicament in the one or more disposable drug units removably coupled to the reusable drive unit; and
adjust the display of the number of injections possible based on a current identification of the medicament in the one or more disposable drug units removably coupled to the reusable drive unit.

27. A method for providing feedback for a rechargeable medication delivery device, the method comprising:
removably coupling one or more disposable drug units to a reusable drive unit of the rechargeable medication delivery device;
performing one or more injections of a dose interval of a medicament to a patient via the one or more disposable drug units removably coupled to the reusable drive unit;
storing, in at least one memory storage element of the reusable drive unit, a power requirement profile for the one or more injections; and
displaying, via at least one indicator icon of the reusable drive unit, a number of injections possible based on a current charge level of a battery of the reusable drive unit and the stored power requirement profile.

28. The method of claim 27, wherein the power requirement profile includes a temperature measurement of the medicament and/or a temperature measurement of the battery.

29. The method of claim 28, further comprising:
adjusting the display of the number of injections possible based on at least one of a current temperature measurement of the medicament or a current temperature measurement of the battery.

30. The method of claim 27, further comprising:
identifying the medicament in the one or more disposable drug units removably coupled to the reusable drive unit.
